(19) United States
(12) Patent Application Publication
(10) Pub. No.: US 2024/0188808 A1
(43) Pub. Date: Jun. 13, 2024

(54) ENDOSCOPE, UNIVERSAL CORD OF ENDOSCOPE, AND METHOD FOR MANUFACTURING UNIVERSAL CORD OF ENDOSCOPE

(71) Applicant: OLYMPUS MEDICAL SYSTEMS CORP., Tokyo (JP)

(72) Inventor: Yoshihiro HIRASAWA, Tokyo (JP)

(73) Assignee: OLYMPUS MEDICAL SYSTEMS CORP., Tokyo (JP)

(21) Appl. No.: 18/530,315

(22) Filed: Dec. 6, 2023

Related U.S. Application Data
(60) Provisional application No. 63/431,363, filed on Dec. 9, 2022.

(51) Int. Cl. A61B 1/005; A61B 1/00

(52) U.S. Cl. CPC A61B 1/0056; A61B 1/00128

(57) ABSTRACT

An endoscope includes an insertion portion, an operation portion located proximally relative to the insertion portion, and a universal cord including a first end connected to the operation portion, a second end including a connector, and a first bending region located between the first end and the second end. A minimum radius of curvature of the first bending region is a first value. The universal cord has a second region adjacent to the first bending region. A minimum radius of curvature of the second region is a second value. The first value is smaller than the second value.

### CROSS REFERENCE TO RELATED APPLICATION

This application is based on and claims priority under 35 U.S.C. § 119 to U.S. Provisional Application No. 63/431,363, filed Dec. 9, 2022, the entire contents of which are incorporated herein by reference.

### BACKGROUND OF THE DISCLOSURE

The disclosure relates to an endoscope, a universal cord of the endoscope, and a method for manufacturing the universal cord of the endoscope.

An endoscope is used to observe a region of a subject that is not visible from the outside by inserting an elongated insertion portion into the subject. A treatment instrument such as forceps is inserted into a channel that passes through the insertion portion of the endoscope. Various treatments are performed using the treatment instrument protruding from a distal end opening of the insertion portion.

Japanese Patent Application Laid-Open Publication No. 2002-45335 discloses an endoscope apparatus in which a region of an insertion portion inserted into a subject is placed in a straight line to undergo high-pressure steam sterilization in order to prevent a reduction in insertability of the insertion portion. The insertion portion other than the region inserted into the subject, and a universal cord provided to extend from an operation portion are placed in the endoscope apparatus in such a manner as to form a large arc.

### SUMMARY OF THE DISCLOSURE

An endoscope according to one embodiment of the disclosure includes: an insertion portion; an operation portion located proximally relative to the insertion portion; and a universal cord including a first end connected to the operation portion, a second end including a connector, and a first bending region located between the first end and the second end. A minimum radius of curvature of the first bending region is a first value. The universal cord has a second region adjacent to the first bending region. A minimum radius of curvature of the second region is a second value. The first value is smaller than the second value.

A universal cord according to another embodiment of the disclosure includes; a connector; a first bending region; and a second region adjacent to the first bending region. A first end of the universal cord is configured to be attached to an operation portion of an endoscope and the connector is located at a second end of the universal cord. A minimum radius of curvature of the first bending region is a first value. A minimum radius of curvature of the second region is a second value. The first value is smaller than the second value.

A method for manufacturing a universal cord of an endoscope according to still another embodiment of the disclosure includes: placing a plurality of internal components to be inserted into a universal cord in a substantially straight line; accommodating a first length section of the plurality of internal components in a first tube portion, the first tube portion having a length approximately a half of an overall length of the universal cord; accommodating a second length section of the plurality of internal components in a second tube portion and connecting the second tube portion to the first tube portion; and accommodating a third length section of the plurality of internal components in a third tube portion and connecting the third tube portion to the second tube portion, a structure of the first tube portion and a structure of the third tube portion are the same, a minimum radius of curvature of the first tube portion is a first value, a minimum radius of curvature of the second tube portion is a second value, and a minimum radius of curvature of the third tube portion is a third value, and the second value is smaller than the first value, and the second value is smaller than the third value.

### BRIEF DESCRIPTION OF THE DRAWINGS

FIG. 1 is a perspective view of a conventional endoscope.

FIG. 2 is a plan view for describing a minimum radius of curvature of a universal cord of the endoscope.

FIG. 3 is a perspective view of an endoscope in a first embodiment.

FIG. 4 is a plan view for describing a minimum radius of curvature of a universal cord of the endoscope in the first embodiment.

FIG. 5 is a partially exploded view for describing a structure of the universal cord of the endoscope in the first embodiment.

FIG. 6 is a view for describing an example of a structure of a first bending region of the universal cord of the endoscope in the first embodiment.

FIG. 7 is a view for describing another example of the structure of the first bending region of the universal cord of the endoscope in the first embodiment.

FIG. 8 is a view for describing still another example of the structure of the first bending region of the universal cord of the endoscope in the first embodiment.

FIG. 9 is a view for describing still another example of the structure of the first bending region of the universal cord of the endoscope in the first embodiment.

FIG. 10A is a view for describing a method for manufacturing the universal cord of the conventional endoscope.

FIG. 10B is a view for describing a method for manufacturing the universal cord of the endoscope in the first embodiment.

FIG. 11 is a perspective view of an endoscope in a first modification of the first embodiment.

FIG. 12 is a perspective view of an endoscope in a second modification of the first embodiment.

FIG. 13 is a perspective view of an endoscope in a second embodiment.

### DETAILED DESCRIPTION

Embodiments and the like of the disclosure are described hereinafter with reference to the drawings.

The drawings are schematic. The relationships between thickness and width of each portion in the drawings, the ratios of thickness among respective portions, and the like are different from the actual relationships, ratios, and the like. Even among the drawings, the dimensional relationships and ratios may differ between some of the portions. Illustrations of, and provision of reference numerals to, some of the components are omitted.

### <Structure of Endoscope>

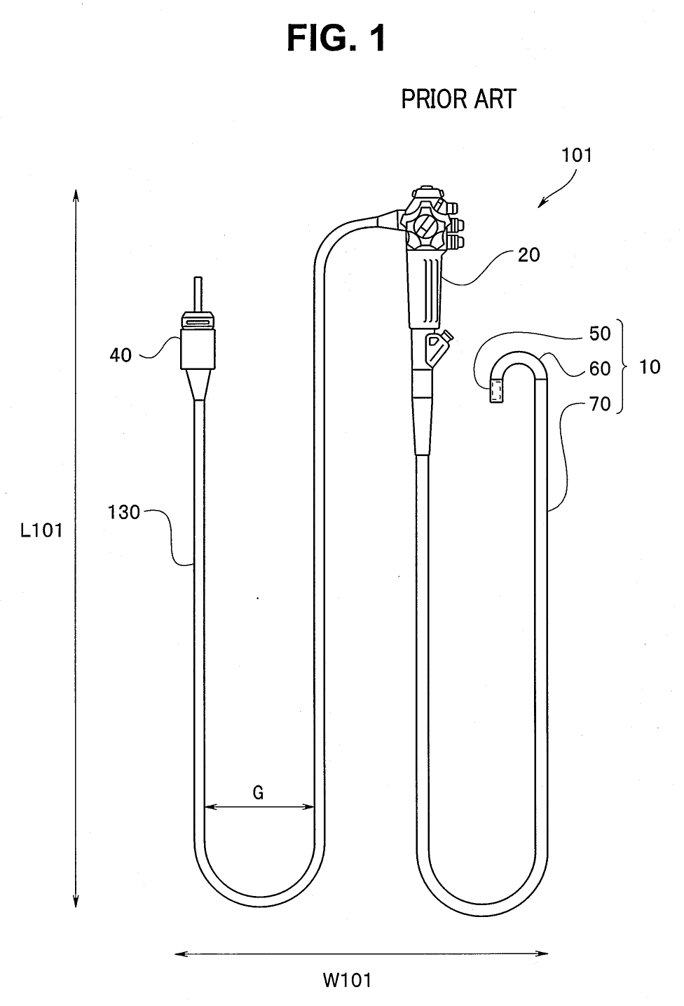

First, an endoscope 101 of a conventional configuration is described. As illustrated in FIG. 1, the endoscope 101 includes an insertion portion 10, an operation portion 20, a universal cord 130, and a connector 40. The insertion portion 10 is inserted into a body of a subject. The operation portion 20 is disposed at a proximal end of the insertion portion 10. The universal cord 130 extends from the operation portion 20. The connector 40 is disposed at a proximal end (second end) of the universal cord 130. The connector 40 is connected to a peripheral device that is not illustrated (such as a processor or a light source).

The insertion portion 10 includes a distal end portion 50 on a distal end side, a bending portion 60 disposed on a proximal end side of the distal end portion 50, and an elongated flexible tube 70 connecting the proximal end side of the bending portion 60 and the operation portion 20. The distal end portion 50, the bending portion 60, and the flexible tube 70 are consecutively provided. An image pickup unit (not illustrated) is disposed at the distal end portion 50. The bending portion 60 bends in, for example, a left-right direction/an up-down direction in response to an operation of the operation portion 20.

The flexible tube 70 and the universal cord 130 have flexibility, and are elastically deformed and bent passively by an external force.

The elongated flexible tube 70 and the elongated universal cord 130 can be stored in a bent state. However, when the flexible tube 70 and the universal cord 130 are bent in a small radius of curvature, the flexible tube 70 and the universal cord 130 may be broken or bent due to so-called shape memory effect. For the above reason, for example, a gap G may be formed as illustrated in FIG. 1 to bend and accommodate the universal cord 130 in a space.

The flexible tube 70 can have a structure that is the same as the structure of the universal cord 130. The same can include substantially the same. Thus, it is not easy to fold and accommodate the flexible tube 70 compactly in the space. The gap G is so wide that a large space with a width W101 and a height L101 may be used to store the endoscope 101 in a bent state. Note that a depth dimension of the space to store the endoscope 101 is determined by the size of the operation portion 20.

In recent years, single-use endoscopes that are disposable after a single use have been utilized. Since a new endoscope is used for each individual subject, a large storage space may be used to store many single-use endoscopes.

When distribution costs should be reduced, the packaging containers used for shipping endoscopes can be downsized.

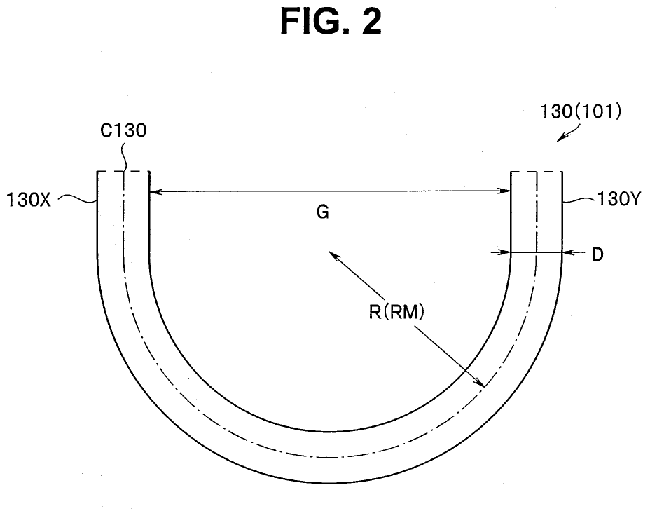

As illustrated in FIG. 2, a radius of curvature R, obtained when a user grasps two regions 130X and 130Y of the universal cord 130 and bends the universal cord 130 in a semicircular shape in such a manner that the two regions 130X and 130Y are parallel and in closest proximity to each other, is hereinafter referred to as “minimum radius of curvature RM.” The radius of curvature R uses a center line C130 of the universal cord 130 as a reference. The minimum radius of curvature RM is a minimum value of the radius of curvature R of the universal cord 130 in a bending state without being plastically deformed.

For example, the minimum radius of curvature RM of the universal cord 130 of the endoscope 101 is two and a half times as large as an outer diameter D of the universal cord 130. Accordingly, the gap G between the two regions 130X and 130Y located parallel to each other on opposite sides of the bending region is approximately four times as large as the outer diameter D of the universal cord 130. For example, provided that the universal cord 130 has the outer diameter D of 15 mm, the gap G is 60 mm.

### First Embodiment

A main configuration of an endoscope 1 in a first embodiment can be substantially the same as the main configuration of the conventional endoscope 101. Thus, in the descriptions below, components of the endoscope 1 having the same functions as the functions of the components of the endoscope 101 are denoted by the same reference numerals, and descriptions of the components are omitted.

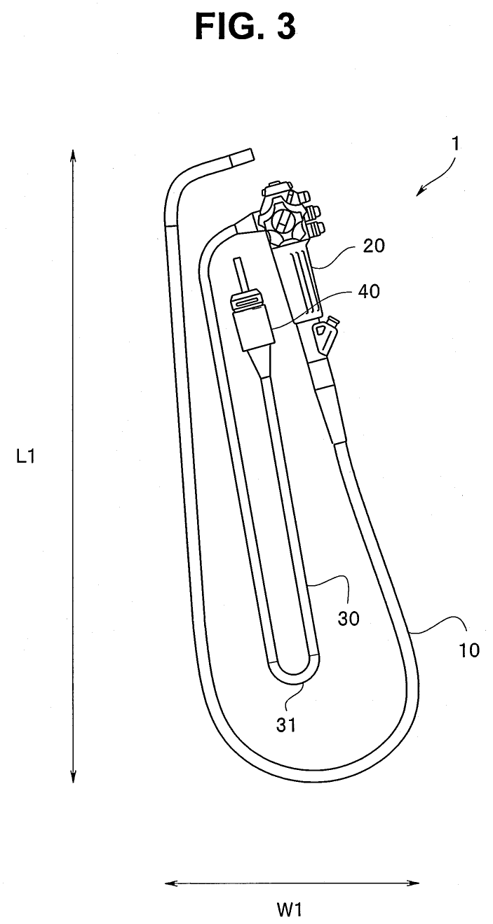

A universal cord 30 of the endoscope 1 in the present embodiment illustrated in FIG. 3 includes a first bending region 31.

As illustrated in FIG. 3, in the endoscope 1, the flexible tube 70 is placed in a state of forming a gentle arc so as to surround the universal cord 30 folded in two.

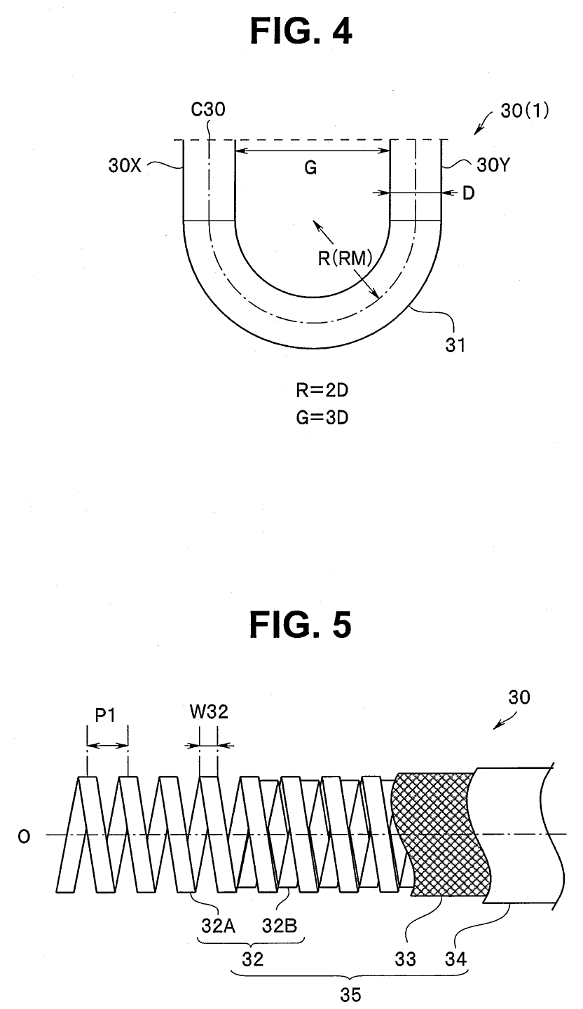

The first bending region 31 has the minimum radius of curvature RM smaller than the minimum radius of curvature RM in an adjacent region. The adjacent region refers to a region other than the first bending region 31 of the universal cord 30, for example, regions 30X and 30Y (FIG. 4) connected to the first bending region 31. The region of the universal cord 30 other than the first bending region 31 has a structure that is the same as, for example, the structure of the universal cord 130 having already been explained.

As illustrated in FIG. 4, in the universal cord 30, the minimum radius of curvature RM of the first bending region 31 is one and a half times (150%) as large as the outer diameter D of the universal cord 30. Accordingly, the gap G between the region 30X and the region 30Y on opposite sides of the first bending region 31 is twice as large as the outer diameter D of the universal cord 30.

Provided that the universal cord 30 has the outer diameter D of 15 mm, the gap G is 30 mm. Note that the minimum radius of curvature RM of the region adjacent to the first bending region 31 is two and a half times as large as the outer diameter D of the universal cord 30, and the gap G is four times as large as the outer diameter D of the universal cord 30.

The first bending region 31 is located at a position at which an overall length L30 of the universal cord 30 is divided approximately into two equal-length parts. Consequently, the universal cord 30 can be folded in a so-called two-folded state and placed in a space of (L1×W1).

Note that the position, at which the overall length L30 of the universal cord 30 is divided approximately into two equal-length parts, refers to a position in the range of greater than 40% and less than 60% of the overall length L30 of the universal cord 30 from an end of the universal cord 30. For example, in the universal cord 30 having the overall length L30 of 1500 mm, the first bending region 31 is located at a position in the range of greater than 600 mm and less than 900 mm from a distal end or a trailing end of the universal cord 30. The first bending region 31 can be at least partially located at the position at which the overall length L30 of the universal cord 30 is divided approximately into two equal-length parts. The center of the first bending region 31 in a length direction can be located at the position at which the overall length L30 of the universal cord 30 is divided approximately into two equal-length parts.

The first bending region 31 can have a length greater than 3% and less than 10% of the overall length L30 of the universal cord 30 in order to ensure operability. When the length of the first bending region 31 is greater than the lower limit of the range described above, it is easy to obtain a desired minimum radius of curvature RM. When the length of the first bending region 31 is less than the upper limit of the range described above, it is possible to suppress an increase in cost and improve ease of storage.

For example, the universal cord 30 can have the overall length L30 of 1500 mm that the first bending region 31 can have a length greater than 45 mm and less than 150 mm.

### <Structure of Universal Cord>

A structure of a main portion of the universal cord 30 can be the same as the structure of the flexible tube 70. The same can include substantially the same.

As illustrated in FIG. 5, the universal cord 30 has an exterior tube 35 in which a spiral tube 32 (flex) is covered with a net-like tube 33 (braid), and the net-like tube 33 is further covered with a resin 34. Although not illustrated, internal components such as an air/water feeding tube, an optical fiber, and a wire are accommodated in the exterior tube 35.

The spiral tube 32 is formed by winding an elongated thin sheet made of metal in a spiral shape around a central axis O in the longitudinal direction of the universal cord 30. The spiral tube 32 is a so-called double flex in which a right-handed first spiral tube 32A and a left-handed second spiral tube 32B are combined. A width W32 of the thin sheet forming the spiral tube 32 is smaller than a winding pitch P1 with which the thin sheet is wound. The spiral tube 32 illustrated in FIG. 5 has the pitch width P1 (P1≥W32×2). Thus, the spiral tube 32 is deformed in response to the bending of the universal cord 30.

The net-like tube 33 in which thin metallic wires are braided is covered with the resin 34 of, for example, fluorine-containing rubber or silicone rubber and having flexibility. Furthermore, the resin 34 may be accommodated in a tube that is highly waterproof and flexible.

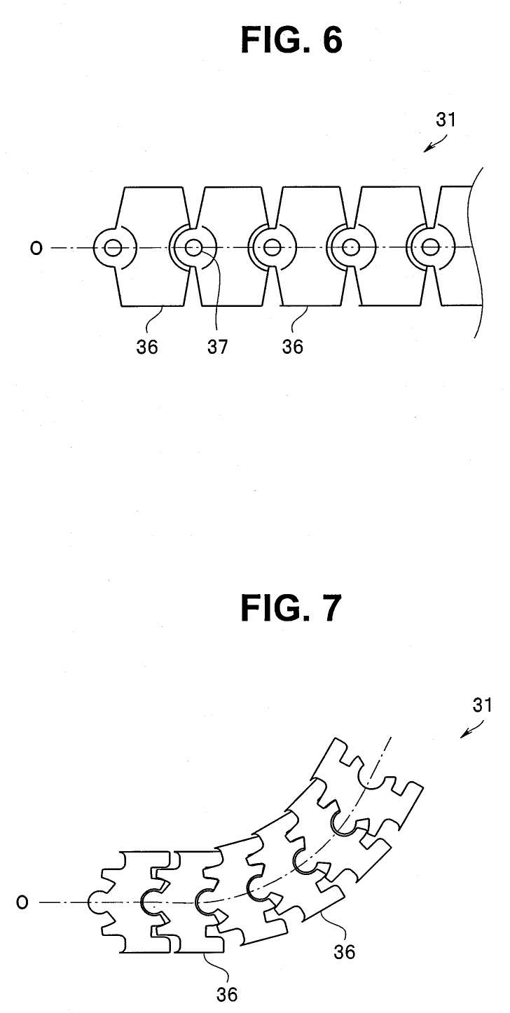

As illustrated in FIG. 6, instead of the spiral tube 32, the first bending region 31 has a structure in which a plurality of bending pieces 36 are consecutively provided in a movable state by using rivets 37. The structure of the first bending region 31 is the same as the structure of the bending portion 60. The same can include substantially the same. However, in contrast to the bending portion 60 that bends by operating an operation wire, the first bending region 31 bends in response to deformation of the universal cord 30. The first bending region 31 not only bends in a smaller radius of curvature, but also bends with a smaller external force due to greater flexibility, compared to the regions 30X and 30Y on opposite sides of the first bending region 31.

The structure of the first bending region 31 is not limited to the above structures.

The first bending region 31 illustrated in FIG. 7 has a bending structure in which a metal tube is divided into the plurality of bending pieces 36 by laser processing. In other words, it is possible for the first bending region 31 to use a structure that is substantially the same as any of various types of structures commonly known as a structure of the bending portion 60, for example, a structure having bending pieces formed by a press forming method.

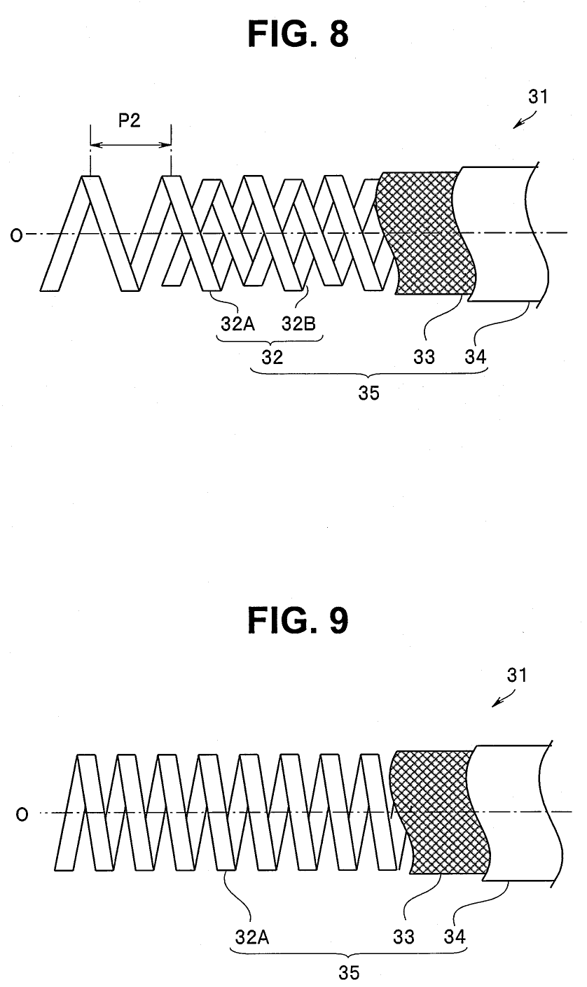

Furthermore, in the first bending region 31 illustrated in FIG. 8, the spiral tube 32 has a winding density smaller than the winding density in the adjacent region. In other words, the structure of the first bending region 31 is the same as the structure of the adjacent region, except that a winding pitch P2 is greater than the winding pitch P1 (FIG. 5) in the adjacent region. In order to achieve a desired minimum radius of curvature, for example, that the winding pitch P2 can be one and a half times greater than the winding pitch P1.

The spiral tube 32 in the first bending region 31 illustrated in FIG. 9 is a single flex made up of only the right-handed first spiral tube 32A. The universal cord 30 adjacent to the first bending region 31 is a double flex. Note that the spiral tube 32 in the first bending region 31 may be a single flex and have a greater winding pitch P than the winding pitch in the adjacent region.

It is allowable that the first bending region 31 does not have a flex, and is made up of only a net-like tube and a resin tube. In the first bending region 31, the flex may be made of thin sheet of resin that is more flexible than metal. The first bending region 31 may be made up of only a resin tube.

As illustrated in FIG. 3, the space with a width W1 and a height L1 for accommodating the endoscope 1 is smaller than the space with the width W101 and the height L101 in which the conventional endoscope 101 is accommodated. Although the endoscope 1 is a single-use endoscope that is disposable after a single use, the endoscope 1 can be accommodated compactly in the space. Consequently, many endoscopes 1 can be stored in a narrow storage space. Transportation cost of the endoscope 1 is low.

The minimum radius of curvature RM of the first bending region can be greater than 50% and less than 250% of the outer diameter D of the universal cord 30. When the minimum radius of curvature RM falls within the range described above, the universal cord 30 can be folded compactly, so that the endoscope can be accommodated compactly in the space.

### <Method for Manufacturing Universal Cord>

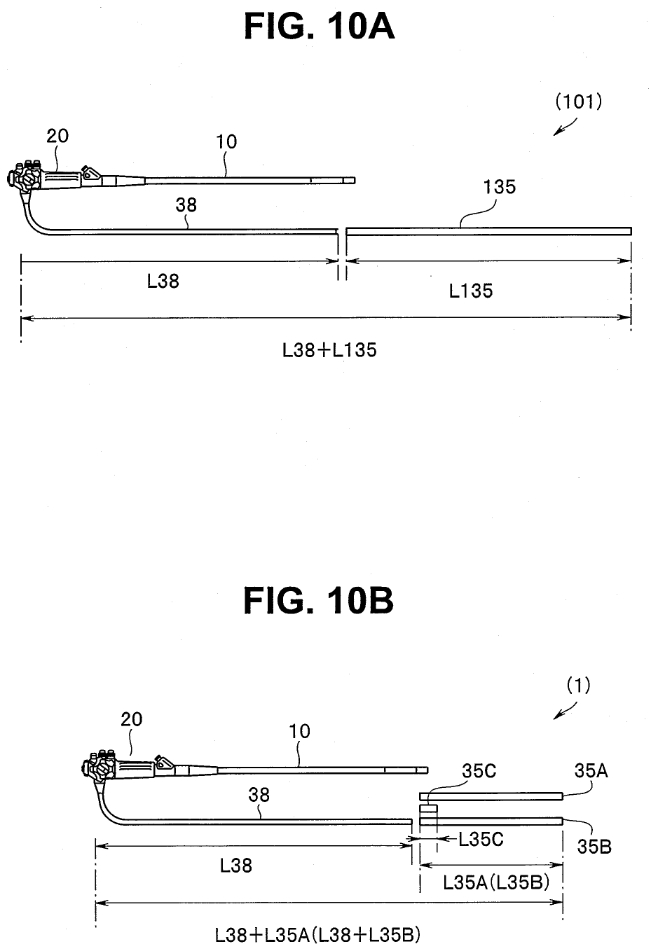

As illustrated in FIG. 10A, in a method for manufacturing the universal cord 130 of the conventional endoscope 101, an internal component 38 (such as an air/water feeding tube, an optical fiber, and a wire) is placed in a straight line on a worktable (not illustrated), and then the internal component 38 is accommodated in the exterior tube 135. The wording “a straight line” refers to a state in which a flexible wire and the like are manually placed at least substantially straightly.

Accordingly, the length of the worktable is the sum of a length L38 of the internal component 38 and a length L135 of the exterior tube 135, so that a large workspace may be used.

In contrast to the conventional endoscope 101, in the endoscope 1 in the embodiment in which the universal cord 30 is foldable in two, an exterior tube 35A (first tube portion or first cylinder) and an exterior tube 35B (third tube portion or third cylinder) are separated from an exterior tube 35C (second tube portion or second cylinder) of the first bending region. The length of the exterior tube 35 can be regarded as the sum of a length L35A of the exterior tube 35A, a length L35B of the exterior tube 35B, and a length L35C of the exterior tube 35C. The reason is that although there is actually a superimposed portion of the exterior tube 35A and the exterior tube 35C, a length of the superimposed portion is relatively smaller than the overall length of the universal cord 30.

Both the length L35A of the exterior tube 35A and the length L35B of the exterior tube 35B can be regarded as approximately half the overall length of the universal cord 30. The reason is that both the length L35A of the exterior tube 35A and the length L35B of the exterior tube 35B are sufficiently greater than the length L35C of the exterior tube 35C. The length L35A of the exterior tube 35A is substantially equal to the length L35B of the exterior tube 35B.

Consequently, as illustrated in FIG. 10B, the length of the worktable is, for example, the sum of the length L38 of the internal component 38 and the length L35A of the exterior tube 35A, so that the universal cord 30 can be manufactured in a small workspace.

The exterior tubes 35A and 35B are connected to the exterior tube 35C by means of welding, soldering, or other connecting methods. A structure of the exterior tube 35A and a structure of the exterior tube 35B can be the same.

As described above, the method for manufacturing a universal cord of the endoscope in the embodiment includes placing a plurality of members to be inserted into the universal cord of the endoscope in a straight line, accommodating the plurality of members in the first tube portion with approximately half the overall length of the universal cord, connecting the second tube portion with a minimum radius of curvature smaller than the minimum radius of curvature of the first tube portion to the first tube portion, so as to accommodate the plurality of members in the second tube portion, and connecting the third tube portion having the same structure as the structure of the first tube portion to the second tube portion, so as to accommodate the plurality of members in the third tube portion.

### Modification of First Embodiment

Endoscopes 1A and 1B in modifications of the first embodiment are similar to the endoscope in the first embodiment. Thus, in the descriptions below, components of the endoscopes 1A and 1B having the same functions as the functions of the components of the endoscope 1 in the first embodiment are denoted by the same reference numerals, and descriptions of the components are omitted.

### First Modification of First Embodiment

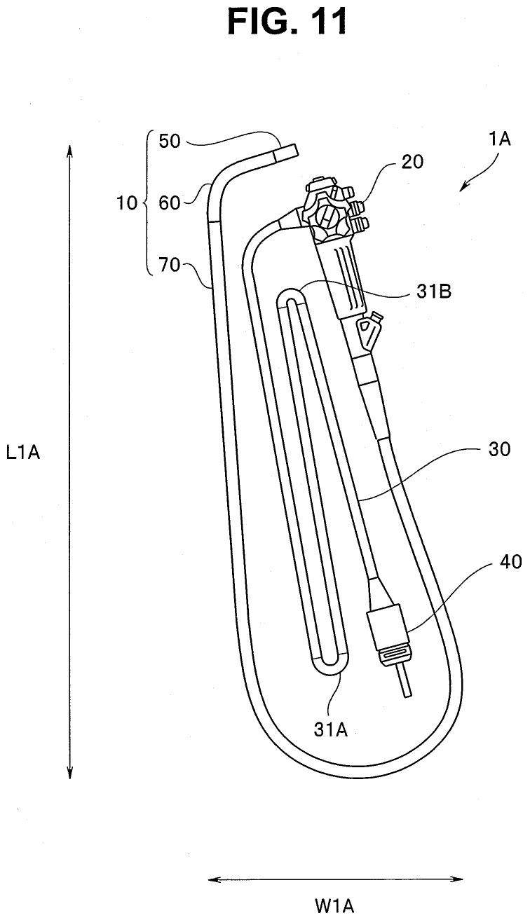

In the endoscope 1A in the present modification illustrated in FIG. 11, the universal cord 30 has first bending regions 31A and 31B at positions at which the overall length L30 is divided approximately into three equal-length parts.

A plurality of first bending regions 31A and 31B may have the same structure or different structures. For example, the first bending region 31A may have the structure illustrated in FIG. 8, and the first bending region 31B may have the structure illustrated in FIG. 9. The plurality of first bending regions 31A and 31B may have different minimum radii of curvature RM.

Since in the endoscope 1A, the universal cord 30 is foldable in three, the endoscope 1A can be accommodated in an even narrower space (W1A×L1A) than the space for the endoscope 1.

The method for manufacturing a universal cord having the first bending regions 31A and 31B can use two cylinders or tubes with approximately one-third of the overall length of the universal cord, and two cylinders or tubes with a length to cover the first bending regions 31A and 31B.

### Second Modification of First Embodiment

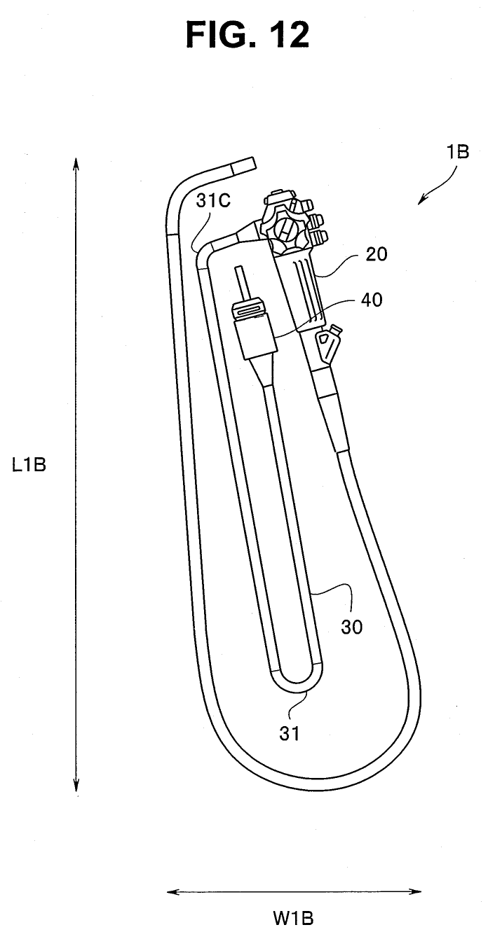

In the endoscope 1B in the present modification illustrated in FIG. 12, a region of the universal cord 30 extending from the operation portion 20 is a first bending region 31C. The first bending region 31C has a structure that is the same as the structure of the first bending region 31A.

The endoscope 1B can be accommodated in an even narrower space (W1B×L1B) than the space for the endoscope 1A.

Note that the universal cord 30 in the embodiment can be folded and compactly accommodated in the space, provided that the universal cord 30 has first bending regions at respective positions at which the overall length is divided into N equal-length parts (N is a natural number larger than 1).

For example, provided that the universal cord 30 has the first bending regions 31 at respective positions at which the overall length L30 is divided approximately into four equal-length parts, the universal cord 30 is fordable into four. However, an endoscope having a large number of first bending regions 31 may result in a reduction in the operability. In view of the possible reduction in the operability, the universal cord 30 can have four first bending regions 31 or less.

### Second Embodiment

An endoscope 1C and components in a second embodiment are similar to the endoscope 1 and components in the first embodiment. Thus, in the descriptions below, the components of the endoscope 1C having the same functions as the functions of the components of the endoscope 1 in the first embodiment are denoted by the same reference numerals, and descriptions of the components are omitted.

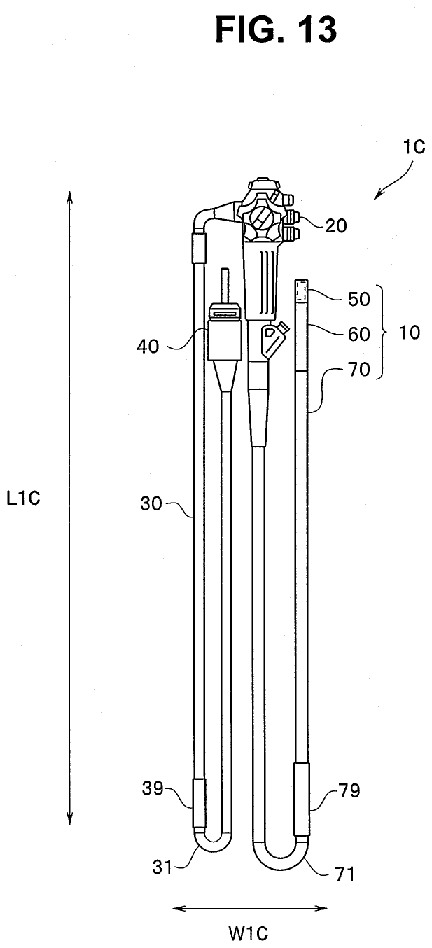

In the endoscope 1C in the present embodiment illustrated in FIG. 13, the flexible tube 70 of the insertion portion 10 has a second bending region 71. The second bending region 71 has a minimum radius of curvature smaller than the minimum radius of curvature in a region adjacent to the second bending region 71. The second bending region 71 has a structure that is the same as any of the various structures of the first bending region 31. The same can include substantially the same.

For example, the second bending region 71 can be located at a position at which the overall length of the flexible tube 70 is divided approximately into equal-length parts or three equal-length parts. The minimum radius of curvature of the second bending region 71 can be greater than 50% and less than 250% of the outer diameter of the flexible tube 70 in order to fold the flexible tube 70 compactly. The second bending region 71 can have a length greater than 4% and less than 15% of the overall length of the flexible tube 70. When the length of the second bending region 71 is greater than the lower limit of the range described above, it is easy to obtain a desired minimum radius of curvature RM. When the length of the second bending region 71 is less than the upper limit of the range described above, the operability of the endoscope is less likely to be reduced.

In the endoscope 1C, not only the universal cord 30, but the flexible tube 70 can also be folded and thus accommodated in a small accommodation space (W1C×L1C).

For example, in an endoscope in which the overall length of the flexible tube 70 is greater than the overall length of the universal cord 30, the flexible tube 70 folded in three and the universal cord 30 folded in two are accommodated in the space. In other words, in the endoscope described above, the universal cord 30 has one first bending region 31, and the flexible tube 70 has two second bending regions 71.

The number of the first bending regions 31 and the second bending regions 71, and the positions where the first bending regions 31 and the second bending regions are individually disposed can be set, such that the length of the universal cord 30 in a folded state is approximately equal to the length of the flexible tube 70 in a folded state.

The endoscope 1C further includes a first tube 39 configured to be attachable to and detachable from and cover at least a portion of the first bending region 31 of the universal cord 30, and a second tube 79 configured to be attachable to and detachable from and cover at least a portion the second bending region 71. For example, the first tube 39 made of elastomer is located adjacent to the first bending region 31 during transportation and storage. At the time of using the endoscope 1C, the first tube 39 is moved to a position where the first tube 39 covers at least a portion of the first bending region 31.

For example, the portion of the first bending region 31 covered with the first tube 39 has a minimum radius of curvature that is substantially equal to the minimum radius of curvature in the adjacent region. The second bending region 71 covered with the second tube 79 has a minimum radius of curvature that is substantially equal to the minimum radius of curvature in the adjacent region. The wording “substantially equal to the minimum radius of curvature” means that the minimum radius of curvature is greater than 60% and less than 130% of the minimum radius of curvature in the adjacent region.

The first tube 39 can have a length greater than 105% and less than 140% of the length of the first bending region 31. The second tube 79 can have a length greater than 105% and less than 140% of the length of the second bending region 71. Provided that both the length of the first tube 39 and the length of the second tube are greater than the lower limit of the range described above, the first tube 39 and the second tube 79 can be easily attached to the first bending region 31 and the second bending region 71, respectively. Provided that both the length of the first tube 39 and the length of the second tube are less than the upper limit of the range described above, the operability is ensured.

The second bending region 71 covered with the second tube 79 can have torque transmission ability and flexibility equivalent to the torque transmission ability and flexibility of the flexible tube 70.

The endoscope 1C having the first tube 39 and the second tube 79 has improved work efficiency because the first bending region 31 and the second bending region 71 do not bend during use. Note that the endoscope may only have either the first tube 39 or the second tube 79. For example, needless to say, an endoscope having a plurality of first bending regions 31 has a plurality of first tubes 39.

In the above embodiments, the endoscope 1 and other endoscopes are single-use endoscopes that are disposable after a single use. However, the endoscope 1 and other endoscopes may be reusable endoscopes that are utilized repeatedly. The endoscope 1 and other endoscopes are not limited to medical use, but may be for industrial use.

The disclosure is not limited to the embodiments and modifications described above. Various changes and modifications can be made without departing from the gist of the disclosure.

It is an object of the embodiments of the disclosure to provide an endoscope that can be compactly accommodated in a space, a universal cord of the endoscope, and a method for manufacturing the universal cord.

According to the embodiments of the disclosure, it is possible to provide an endoscope that can be compactly accommodated in a space, a universal cord of the endoscope, and a method for manufacturing the universal cord.

What is claimed is:

1. An endoscope, comprising:
an insertion portion;
an operation portion located proximally relative to the insertion portion; and
a universal cord including a first end connected to the operation portion, a second end including a connector, and a first bending region located between the first end and the second end,
wherein a minimum radius of curvature of the first bending region is a first value,
wherein the universal cord has a second region adjacent to the first bending region and a minimum radius of curvature of the second region is a second value, and
wherein the first value is smaller than the second value.

2. The endoscope according to claim 1, wherein the first bending region is located at a position at which an overall length of the universal cord is divided approximately into two equal-length parts.

3. The endoscope according to claim 1, wherein the first bending region is located at a position at which an overall length of the universal cord is divided approximately into three equal-length parts.

4. The endoscope according to claim 1, wherein the first value is greater than 50% and less than 250% of an outer diameter of the universal cord.

5. The endoscope according to claim 1, wherein the first bending region has a length greater than 3% and less than 10% of an overall length of the universal cord.

6. The endoscope according to claim 1, wherein the insertion portion has a distal end portion, a bending portion, and a flexible tube,
wherein a structure of the first bending region is the same as a structure of the bending portion, and
wherein the structure of the first bending region is different from a structure of the flexible tube.

7. The endoscope according to claim 1, wherein the universal cord includes a spiral tube formed by winding an elongated thin sheet in a spiral shape,
wherein, in the first bending region, the spiral tube has a first winding density,
wherein, in the second region, the spiral tube has a second winding density, and
wherein the first winding density is smaller than the second winding.

8. The endoscope according to claim 1, wherein an end of the first bending region is connected to the operation portion.

9. The endoscope according to claim 1, wherein the insertion portion has a distal end portion, a bending portion, and a flexible tube,
wherein the flexible tube has a second bending region,
wherein a minimum radius of curvature of the second bending region is a third value, and
wherein the third value is smaller than the second value.

10. The endoscope according to claim 1, further comprising a first tube attachable to and detachable from the first bending region,
wherein a portion of the first bending region is covered with the first tube,
wherein a minimum radius of curvature of the portion of the first bending region covered with the first tube is a fourth value, and
wherein the fourth value is substantially equal to the second value.

11. The endoscope according to claim 9, further comprising a second tube attachable to and detachable from the second bending region,
wherein a portion of the second bending region is covered with the second tube,
wherein a minimum radius of curvature of the portion of the second bending region covered with the second tube is a fifth value, and
wherein the fifth value is substantially equal to the second value.

12. The endoscope according to claim 11, wherein the second bending region covered with the second tube has a flexibility that is substantially the same as a flexibility of the flexible tube.

13. A universal cord, comprising:
a connector;
a first bending region; and
a second region adjacent to the first bending region,
wherein a first end of the universal cord is configured to be attached to an operation portion of an endoscope and the connector is located at a second end of the universal cord,
wherein a minimum radius of curvature of the first bending region is a first value,
wherein a minimum radius of curvature of the second region is a second value, and
wherein the first value is smaller than the second value.

14. A method for manufacturing a universal cord of an endoscope, the method comprising:
placing a plurality of internal components to be inserted into the universal cord in a straight line;
accommodating a first length section of the plurality of internal components in a first tube portion, the first tube portion having a length approximately a half of an overall length of the universal cord;
accommodating a second length section of the plurality of internal components in a second tube portion and connecting the second tube portion to the first tube portion; and
accommodating a third length section of the plurality of internal components in a third tube portion and connecting the third tube portion to the second tube portion,
wherein a structure of the first tube portion and a structure of the third tube portion are the same,
wherein a minimum radius of curvature of the first tube portion is a first value, a minimum radius of curvature of the second tube portion is a second value, and a minimum radius of curvature of the third tube portion is a third value, and
wherein the second value is smaller than the first value, and the second value is smaller than the third value.

15. The method according to claim 14, wherein the first value is the same as the third value.

16. The method according to claim 14, wherein, when the first tube portion is bent to have the minimum radius of curvature of the first value, the first tube portion is not plastically deformed,
wherein, when the second tube portion is bent to have the minimum radius of curvature of the second value, the second tube portion is not plastically deformed, and
wherein, when the third tube portion is bent to have the minimum radius of curvature of the third value, the third tube portion is not plastically deformed.

17. The endoscope according to claim 1, wherein the minimum radius of curvature of the first bending region is a minimum value of a radius of curvature of the first bending region in a bent state at which the first bending region is not plastically deformed.

18. The endoscope according to claim 17, wherein the minimum radius of curvature of the second region is a minimum value of a radius of curvature of the second region in a bent state at which the second region is not plastically deformed.

19. The endoscope according to claim 1, wherein the first bending region comprises a plurality of bending pieces.

20. The endoscope according to claim 19, wherein adjacent bending pieces in the plurality of bending pieces are connected together by a plurality of rivets forming a pivot axis.
